(19) United States
(12) Patent Application Publication
(10) Pub. No.: US 2010/0028967 A1
(43) Pub. Date: Feb. 4, 2010

(54) Method of changing property or function of substance and method of eliminating biofunction of cell

(76) Inventors: Kazuo Nishikawa, Higashiosaka-Shi (JP); Hisaharu Yagi, Kizugawa-Shi (JP); Ai Tsutsui, Kyoto (JP)

Correspondence Address: BIRCH STEWART KOLASCH & BIRCH, PO BOX 747, FALLS CHURCH, VA, 22040-0747, US

(21) Appl. No.: 11/661,379

(22) PCT Filed: May 26, 2005
(86) PCT No.: PCT/JP05/09647

§ 371 Date: Feb. 28, 2007

(30) Foreign Application Priority Data
Sep. 8, 2004 (JP) 2004-261360

(51) Int. Cl. C12N 13/00; C07K 1/107

(52) U.S. Cl. 435/173.1; 530/402

(57) ABSTRACT

There are provided a method of changing a property or function of a substance by fragmenting a protein contained in the substance floating in a gas, and a method of eliminating a biofunction of a cell by applying to the cell either an electric discharge or a particle produced by the electric discharge, or both simultaneously, to fragment a protein contained in the cell.

### TECHNICAL FIELD

The present invention relates to a method of changing a property or function of a substance and a method of eliminating a biofunction of a cell, by fragmenting a protein.

### BACKGROUND ART

Conventionally, a protein can be denatured or degraded by oxidation or reduction treatment by a chemical agent, degradation by an enzyme, or the like.

For example, Japanese Patent Laying-Open No. 06-098791 describes that a gaseous molecule of a sublimation antimicrobial agent A-BCA is diffused and comes into contact with a microorganism on an internal wall of an air conditioner body to destroy a cell membrane and denature a protein of the microorganism, thereby preventing growth of the microorganism on the internal wall of the air conditioner body.

Further, Japanese Patent Laying-Open No. 06-098791 describes a method of cleaving a protein by enzymolysis treating the protein in a controlled or limited manner to obtain an enzyme or a protein fragment, by treating the protein with a protease in the presence of either a surface-active agent or a chaotropic substance other than a salt having coagulation activity.

Furthermore, for example in Japanese Patent Laying-Open No. 63-156950, papain and chymotrypsin are described as proteolytic enzymes.

However, of the conventional methods described above, in the method using a chemical agent, there is no chemical agent currently in use which can widely and completely sterilize all the viruses and bacteria, and each chemical agent is required to have a specific concentration and exposure time to ensure effective sterilization.

In the method using an enzyme, external circumstances such as temperature, humidity, and illuminance should be controlled to cause an enzyme reaction. For example, generally the reaction proceeds only at a body temperature (36-37° C.), and thus the enzyme reaction does not occur in a general living environment.

On the other hand, since denaturation of a protein is performed merely by oxidizing or reducing a portion thereof, there may be a case where a biological defense function of a cell or a microorganism is activated, for example an antioxidant such as a catalase is secreted to suppress an oxidation reaction or repair a protein. In such a case, a sufficient effect cannot be obtained.

Patent Document 1: Japanese Patent Laying-Open No. 06-098791

Patent Document 2: Japanese Patent Laying-Open No. 63-156950

### DISCLOSURE OF THE INVENTION

### Problems to be Solved by the Invention

The present invention has been made in view of the above circumstances, and one object of the present invention is to provide a method of changing a property or function of a substance having a protein and eliminating a biofunction of a cell or a microorganism, by fragmenting the protein.

### Means for Solving the Problems

According to one aspect of the present invention, there is provided a method of changing a property or function of a substance by fragmenting a protein contained in the substance floating in a gas.

Preferably, the substance is a granular substance, a microorganism, or a cell.

Preferably, during the fragmenting, a particle having reactivity is applied to the substance.

Preferably, during the fragmenting, an electric discharge or a particle produced by the electric discharge is applied to the substance, or both are simultaneously applied to the substance.

Preferably, the particle described above includes a charged particle or an excited particle, and when the particle produced by an electric discharge is applied, either one or both of the charged particle and the excited particle are released to the substance.

Preferably, the charged particle includes a positive ion and a negative ion, the positive ion being H⁺(H₂O)ₙ (where n is a natural number), and the negative ion being O₂⁻(H₂O)ₘ (where n is a natural number).

Preferably, the positive ion and the negative ion have a total concentration in a range of 10000 ions/cm³ to 1000000 ions/cm³.

Preferably, the particle produced by the electric discharge includes a hydroxy radical.

According to another aspect of the present invention, there is provided a method of eliminating a biofunction of a cell by applying to the cell either an electric discharge or a particle produced by the electric discharge, or both simultaneously, to fragment a protein contained in the cell.

Preferably, the particle produced by the electric discharge includes a charged particle or an excited particle, and when the particle produced by the electric discharge is applied, either one or both of the charged particle and the excited particle are released to the cell.

Preferably, the charged particle includes a positive ion and a negative ion, the positive ion being H⁺(H₂O)ₙ (where n is a natural number), and the negative ion being O₂⁻(H₂O)ₘ (where m is a natural number).

Preferably, the positive ion and the negative ion have a total concentration in a range of 10000 ions/cm³ to 1000000 ions/cm³.

Preferably, the particle produced by the electric discharge includes a hydroxy radical.

Preferably, the cell is a cell of a microorganism.

Preferably, the applying is performed in a gas.

### EFFECTS OF THE INVENTION

According to the present invention, by fragmenting a protein, a property or function of a substance having the protein can be changed, and a biofunction of a cell can be eliminated.

### BRIEF DESCRIPTION OF THE DRAWINGS

FIG. 1 is a perspective view of an apparatus to be used for the method of the present invention.

FIG. 2 shows, in graphs, the relationship between mass number and ion intensity, in which (a) depicts the case of a positive ion, and (b) depicts the case of a negative ion.

FIG. 3 shows, in a graph, the relationship between wavelength and absorbance for identifying an excited particle.

FIG. 4 shows, in a graph, the relationship between mass change in a protein and ion release time.

FIG. 5 shows, in a graph, the relationship between relative concentration and ion release time in a 34-kDa protein and a 94-kDa protein.

FIG. 6 shows protein density distribution on the surface of a bacterium when positive and negative ions are released and when the ions are not released.

FIG. 7 is a schematic view of an apparatus used for an example.

FIG. 8 shows, in a graph, the relationship between ion release time and survival rate for various bacteria.

FIG. 9 shows, in a graph, the relationship between ion release time and CFU (colony forming unit) for Penicillum chrysogenum.

FIG. 10 shows, in a graph, the relationship between ion release time and CFU for Stachybotrys chartarum.

FIG. 11 shows, in a graph, the relationship between ion release time and CFU for Aspergillus versicolor.

FIG. 12 shows, in a graph, the relationship between ion release time and CFU for Penicillum camambertii.

FIG. 13 shows, in a graph, the relationship between ion release time and CFU for Cladosporium herbarum.

FIG. 14 shows the states of Cladosporium herbarum and Aspergillus versicolor when the ions are not released and when four hours have passed after the ion release.

FIG. 15 is a schematic view of an apparatus used for the present example.

FIG. 16 shows absorbances of Cry j 1 and Cry j 2 when treated and not treated with the ions.

FIG. 17 shows, in a graph, the relationship between ion release time and survival rate for Micrococcus roseus when cold preservation is performed and not performed.

FIG. 18 shows, in a graph, the relationship between ion release time and survival rate for Enterococcus malodoratus when cold preservation is performed and not performed.

FIG. 19 shows, in a graph, the relationship between ion release time and survival rate for Staphylococcus chromogenes when cold preservation is performed and not performed.

FIG. 20 shows, in a graph, the relationship between ion release time and survival rate for Sarcina flava when cold preservation is performed and not performed.

### DESCRIPTION OF THE REFERENCE SIGNS

101 discharge electrode, 102 alumina dielectric, 103 counter electrode, 104 high-voltage pulse power source, 601, 1021 discharge apparatus, 602 tray, 603 PBS buffer, 604 arrow, 605 box, 1022 positive ion, 1023 negative ion, 1024 nebulizer, 1025 collection receptacle, 1026 gas outlet, 1027 closed container, 1028 inlet.

### BEST MODES FOR CARRYING OUT THE INVENTION

The present invention provides a method of changing a property or function of a substance by fragmenting a protein contained in the substance floating in a gas.

In particular, the present invention has a characteristic that a protein can be fragmented in a state floating in a gas, without using a chemical or an enzyme, and thereby a property or function of a substance or particle containing the protein can be changed. According to such a method of the present invention, there is no restriction on concentration, time, humidity, or the like required when a protein is denatured or degraded using a chemical or an enzyme. Further, when a protein is degraded using a chemical or the like, it is necessary to bring the protein and the chemical or the like into contact with each other. Since the chemical or the enzyme generally exists as a liquid in its normal state, it is necessary to immerse the protein in the liquid or bring the protein into contact with the liquid. In contrast, the present invention has an excellent advantage that a protein can be fragmented in a gas, and thus there is no need to immerse a substance or the like containing a protein in a liquid.

Fragmentation of a protein means to cleave one or a plurality of bonds in a molecular frame constituting the protein, by which the protein is structurally separated. Fragmentation of a protein includes cleavage of a bond in a molecular frame constituting the protein by chemical modification. By structurally separating a protein in this manner, the molecular weight of the protein is changed, in particular reduced, and the property and function of a substance containing the protein are changed, in particular eliminated.

Further, the wording “floating in a gas” includes not only the case where a substance is floating in a gas but also the case where a substance adheres to or is in contact with an object in a gas.

In the present invention, a substance means a material existing in a space in a three-dimensional manner. In particular, the present invention is directed to a substance partly or entirely containing a protein. For example, in the present invention, the substance may be granular, and may include a bacterium, a cell, or the like.

Further, the present invention provides a method of eliminating a biofunction of a cell by applying to the cell either an electric discharge or a particle produced by the electric discharge, or both simultaneously, to fragment a protein contained in the cell.

By applying to a cell either one or both of an electric discharge and a particle produced by the electric discharge, a surface membrane protein of the cell is fragmented. Thereby, a hole or rupture is produced in the surface membrane protein, and thus the cell cannot maintain a normal form, losing its biofunction.

### (Method of Fragmenting a Protein)

The method of fragmenting a protein in the present invention is to fragment a protein contained in a substance floating in a gas. As means for that purpose, air including the substance containing the protein is passed through a region in which a predetermined electric discharge is supplied, and the substance is subjected to either the electric discharge or a particle produced by the electric discharge, or both simultaneously. The particle produced by the electric discharge can also be applied to a target substance by releasing the particle to the target substance, or by releasing the particle to a space including the target substance so as to follow the flow of air. Such an electric discharge can be supplied for example by an apparatus described later.

In the present invention, the particle produced by the electric discharge includes a charged particle or an excited particle, and the method of fragmenting a protein also includes delivering either the charged particle or the excited particle, or both simultaneously, into a gas and releasing one or both of them to a floating substance or particle. It is to be noted that a charged particle refers to a particle charged or ionized by an electric discharge, and an excited particle refers to a particle excited by an electric discharge.

### (Method of Eliminating a Biofunction of a Cell)

Further, the present invention provides a method of eliminating a biofunction of a cell by applying to the cell either an electric discharge or a particle produced by the electric discharge, or both simultaneously, to fragment a protein contained in the cell.

Generally, a surface membrane protein exists on the surface of a cell. By fragmenting the surface membrane protein, a hole or rupture is produced in the surface membrane protein, and thus the cell cannot maintain a normal form. As a result, the cell loses its biofunction, in particular, the cell is dead.

Techniques of applying to a cell either an electric discharge or a particle produced by the electric discharge include a technique of applying to a cell either an electric discharge or a particle produced by the electric discharge, or both simultaneously, by causing the cell to pass through a region in which a predetermined electric charge is supplied, and a technique of delivering a particle produced by an electric discharge into a gas and releasing it to a cell. In this case, the particle produced by the electric discharge includes a charged particle or an excited particle, and either the charged particle or the excited particle can be released, or both can be released simultaneously.

The cell described above includes a cell of a microorganism. By fragmenting a protein contained in the microorganism, the cell surface membrane protein of the microorganism is fragmented, and a hole or rupture is produced in the cell membrane. This causes malfunction of the cell membrane of a bacterium, and death of the bacterium.

### (Apparatus)

In the present invention, a discharge apparatus provides an electric discharge or a particle produced by the electric discharge capable of fragmenting a protein. Although the location for installing such a discharge apparatus is not specifically limited, generally it is preferably installed in a region in which it can perform an electric discharge function to a target protein-containing substance or cell.

Since the particle produced by the electric discharge disappears in a short period of time, the discharge apparatus installed as described above can efficiently diffuse these particles in the air. The number of the discharge apparatus to be installed may be one, or two or more.

As such a discharge apparatus, a conventionally known apparatus can be used. Examples of the discharge apparatus include, but not limited to, a surface discharge device, a corona discharge device, a plasma discharge device.

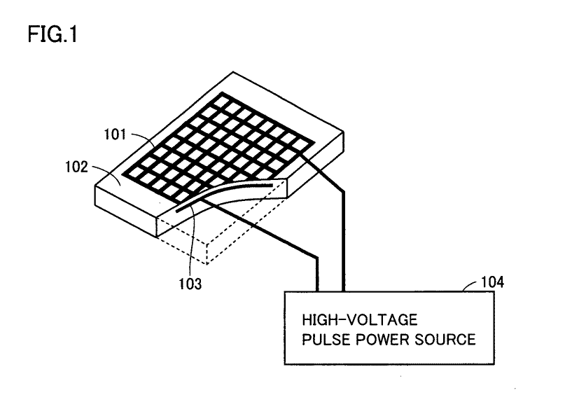

FIG. 1 shows an example of the apparatus capable of generating an electric discharge or a particle produced by the electric discharge in the present invention. FIG. 1 is a perspective view of an apparatus to be used for the method of the present invention. In FIG. 1, a discharge electrode 101 is disposed on a surface of an alumina dielectric 102, and a counter electrode 103 is embedded within alumina dielectric 102 to serve as a discharging portion. Further, a high-voltage pulse power source 104 is connected to the discharge electrode and the counter electrode.

Preferably, in FIG. 1, the distance between the above two electrodes can be about 0.2 mm, discharge electrode 101 forms a mesh pattern, and the discharge electrode has a shape of a rectangle with a size of about 1 cm×3 cm.

In FIG. 1, positive and negative high-voltage pulse voltages (with a frequency of 60 Hz and a peak voltage of about 2 kV) are generated from high-voltage pulse power source 104, and applied across the electrodes to generate an electric discharge.

Further, in a case where a voltage-applying electrode has a shape of a plate or a mesh and a ground-side electrode has a shape of a mesh in such a discharge apparatus, when a high voltage is applied, an electric field is concentrated at a mesh end surface portion of the ground-side electrode and surface discharge is caused, forming a plasma region. When air is flown into the plasma region, not only a particle is produced, but also electrical impact by plasma can be provided.

Although electric discharge is generally performed in the present invention by alternately applying positive and negative voltages, it is also possible to apply one of the positive and negative voltages for a predetermined period of time, and subsequently apply the other voltage for a predetermined period of time.

Further, the shape or material of the electrodes of the discharge apparatus is also not limited to the above, and any shape or material can be selected, including the shape of a needle.

In order to apply to a substance an electric discharge or a particle produced by the electric discharge, such an apparatus preferably has a mechanism to deliver the particle into the air, although not shown. For example, the apparatus can be equipped with an air controlling mechanism. It is to be noted that an air controlling mechanism refers to a mechanism controlling normal air, such as a mechanism provided in an air controlling apparatus such as an air purifier, an air conditioner, a dehumidifier, a humidifier, an electric heater, a kerosene stove, a gas heater, a cooler box, and a refrigerator.

### (Charged Particle)

In the discharge apparatus as shown in FIG. 1, a positive ion and a negative ion are produced as a result of an electric discharge phenomenon applying positive and negative voltages alternately. Of the produced positive and negative ions, the positive ion has a structure of H⁺(H₂O)ₙ (where n is an arbitrary natural number), which is formed by ionizing a water molecule in the air by plasma discharge to produce a hydrogen ion H⁺, and clustering water molecules in the air around the hydrogen ion using solvation energy.

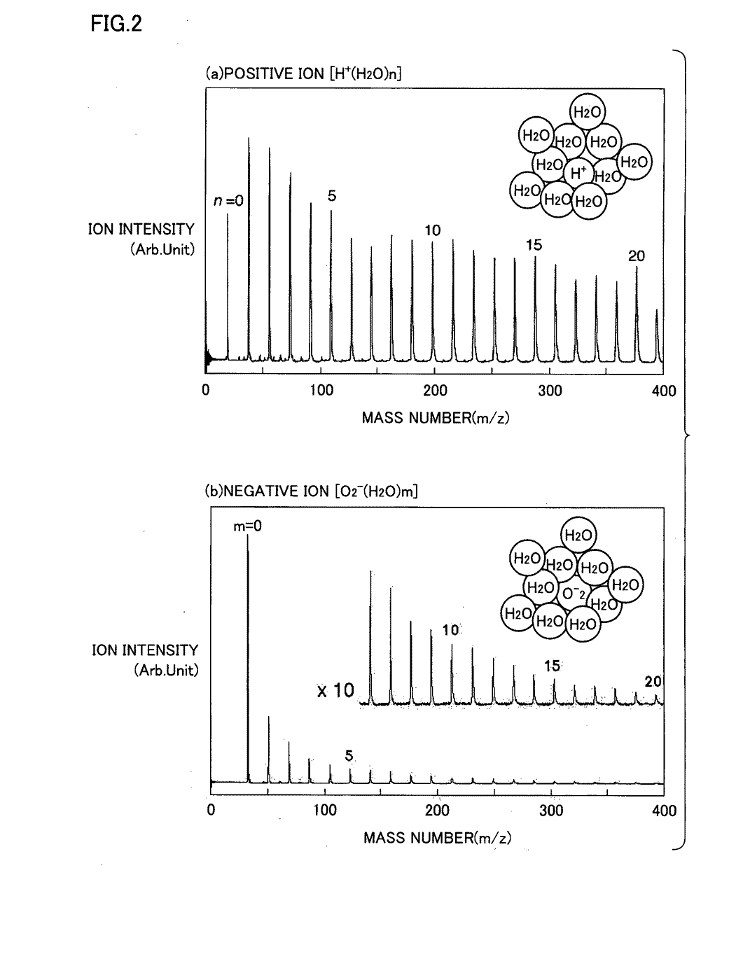

The clustering of water molecules is clear from FIG. 2. Specifically, the clustering of water molecules is clear from FIG. 2(a), in which a peak observed at the minimum is located at the position with a molecular weight of 19, and subsequent peaks appear at positions with molecular weights obtained by sequentially adding 18 (the molecular weight of water) to the molecular weight of 19. That is, this result shows that a hydrogen ion H⁺ with a molecular weight of 1 is hydrated with water molecules with a molecular weight of 18 in a cluster.

On the other hand, the negative ion has a structure of O₂⁻(H₂O)ₘ (where m is an arbitrary natural number), which is formed by ionizing an oxygen molecule or a water molecule in the air by plasma discharge to produce an oxygen ion O₂⁻, and clustering water molecules in the air around the oxygen ion using solvation energy.

The clustering of water molecules is clear from FIG. 2(b), in which a peak observed at the minimum is located at the position with a molecular weight of 32, and subsequent peaks appear at positions with molecular weights obtained by sequentially adding 18 (the molecular weight of water) to the molecular weight of 32. That is, this result shows that an oxygen ion O₂⁻ with a molecular weight of 32 is hydrated with water molecules with a molecular weight of 18 in a cluster.

In the present invention, the charged particle produced by the electric discharge includes H⁺(H₂O)ₙ and O₂⁻(H₂O)ₘ (where n and m are arbitrary natural numbers) as described above.

### (Excited Particle)

In the present invention, the excited particle is produced as a result of the above electric discharge phenomenon. The excited particle is mainly composed of a hydroxy radical (.OH) produced by dissociating a water molecule in the air by plasma discharge. Further, when the charged particle described above is delivered into a space, these positive and negative ions surround a substance or particle floating in the air, and the positive and negative ions produce on a surface a hydroxy radical (.OH), which is an active species, according to the following chemical reaction (1):

H₃O⁺+O₂⁻→3.OH (1).

In the present invention, the excited particle also includes a hydroxy radical produced by such a reaction of the positive and negative ions in the charged particle.

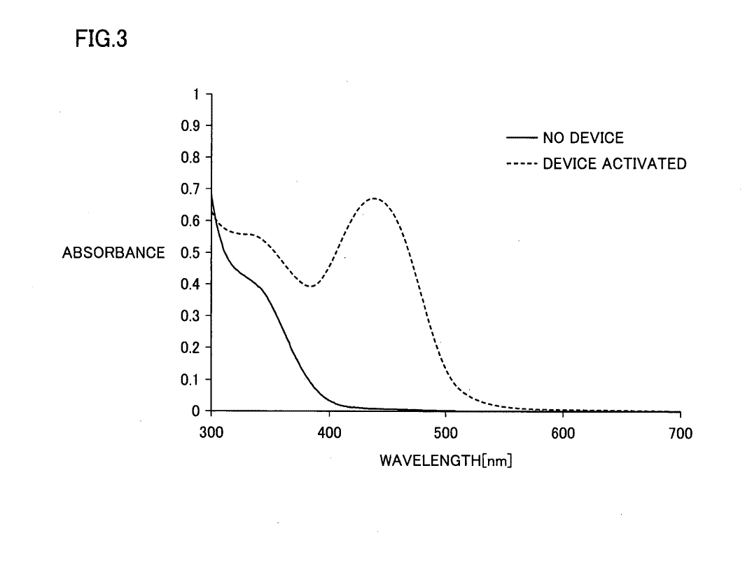

In order to identify an excited particle produced by electric discharge, reaction absorbance analysis was performed in the present invention, using a WST-1(2-(4-iodophenyl)-3-(4-nitrophenyl)-5-(2,4-disulfophenyl)-2H-tetrazolium, monosodium salt) reagent. FIG. 3 shows the result.

In FIG. 3, the axis of ordinate represents absorbance, and the axis of abscissa represents wavelength. As clear from FIG. 3, an absorption spectrum generated by the reaction between the WST-1 reagent and superoxide (.O) and hydrogen peroxide (H₂O₂) was not confirmed, and when electric discharge was performed, an absorption spectrum generated by the reaction between the WST-1 reagent and a hydroxy radical (.OH) was confirmed at around a wavelength of 430 nm, as compared with the case where electric discharge was not performed. That is, this result shows that only the hydroxy radical (.OH) was produced.

### (Ion Concentration)

In the present invention, the positive and negative ions in the charged particle are preferably released with a total concentration in a range of 10000 ions/cm³ to 1000000 ions/cm³. If the total concentration is less than 10000 ions/cm³, it is difficult to achieve a satisfactory result due to the small number of the ions. For example, if the ions are released with an ion concentration of 800 ions/cm³, it is difficult to achieve a significant reduction in reactivity with a monoclonal antibody. Further, if the total concentration exceeds 1000000 ions/cm³, electric discharge intensity is increased, and thus electric discharge by-products such as ozone and nitrogen oxide may be produced. Thereby, ozone concentration may exceed the industrial hygienic standard of 0.1 ppm, which is problematic. More preferably, the total concentration is not less than 11500 ions/cm³ and not more than 12050 ions/cm³.

The ion concentration can be adjusted by adjusting an applied voltage, or by blowing air to suppress adhesion of the ions and diffuse the ions. Further, the ion concentration can be confirmed by a Gerdien capacitor.

### EXAMPLES

Hereinafter, examples will be given to describe the present invention in more detail. However, the present invention is not limited to these examples.

### Example 1

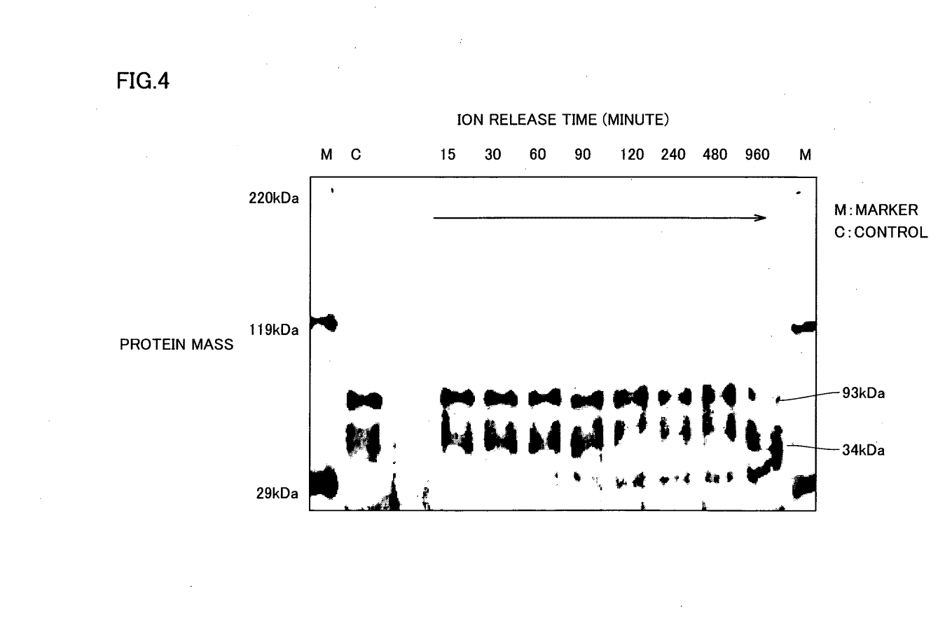

An adhesive bacterium was used as a substance containing a protein, or a cell, and air containing positive and negative ions as particles produced by an electric discharge was released to the adhesive bacterium, in order to examine the change in the protein. Specifically, positive and negative ions were released to an Enterococcus bacterium, and a membrane protein was extracted at each release time to measure mass distribution of the protein by SDS-Page electrophoresis. FIG. 4 shows the result. In FIG. 4, the axis of ordinate represents mass of the protein, and the axis of abscissa represents time to release the positive and negative ions. It is to be noted that, in FIG. 4, the leftmost bar represents a marker, and the second leftmost bar represents a control.

As seen in FIG. 4, during the passage of ion release time, fluorescence disappeared at a mass of 93 kDa, and appeared at a mass of 34 kDa. Further, for the protein with a mass of 93 kDa and the protein with a mass of 34 kDa, change in concentration over time was examined. As shown in FIG. 5, the protein with a mass of 93 kDa decreased, and the protein with a mass of 34 kDa increased. This is likely to indicate that the protein with a mass of 93 kDa was fragmented into the protein with a mass of 34 kDa.

From the above result, it has been found that, according to the method of the present invention, a substance containing a protein, specifically a protein in a cell is fragmented by an electric discharge or a particle produced by the electric discharge.

### Example 2

An Enterococcus bacterium adhered to an agar medium was used as a substance containing a protein, specifically a cell, and positive and negative ions as particles produced by an electric discharge were released to the Enterococcus bacterium for three hours, in order to examine the change in the protein in the Enterococcus bacterium.

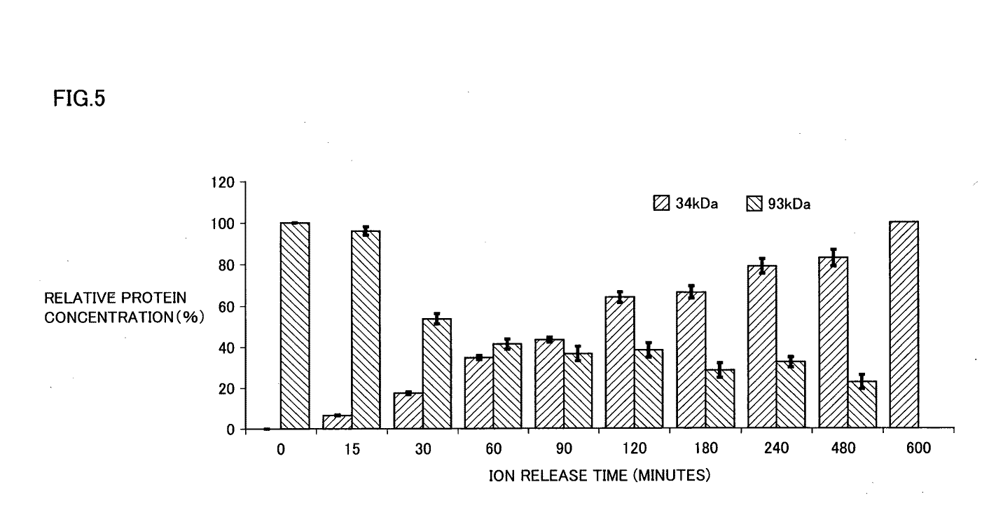

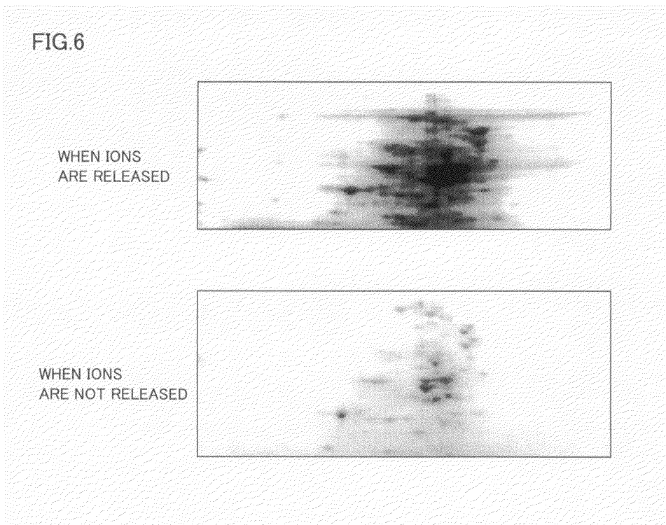

As an experimental technique, analysis by two-dimensional SDS-Page electrophoresis was employed. Isoelectric focusing electrophoresis was performed with a gradient of pH 3 to 10. A gel was stained with Coomassie Blue stain (PageBlue by Fermentas) and introduced, and thereafter image analysis was performed on density distribution. FIG. 6 shows density distribution of the protein on the surface of the above bacterium when positive and negative ions were released and when the ions were not released.

In FIG. 6, a portion having a dark black shadow indicates that a membrane protein exists, and a portion having a light black shadow indicates that the membrane protein disappears. This result shows that the membrane protein is fragmented by the release of positive and negative ions, and a hole is produced in the membrane on the surface of the bacterium. It has become clear that fragmentation of the membrane protein on the surface of the bacterium causes malfunction of the membrane and death of the bacterium.

From the above result, it has been found that, according to the method of the present invention, a membrane protein of a cell is fragmented and a biofunction of the cell is eliminated, in particular, the cell is dead.

### Example 3

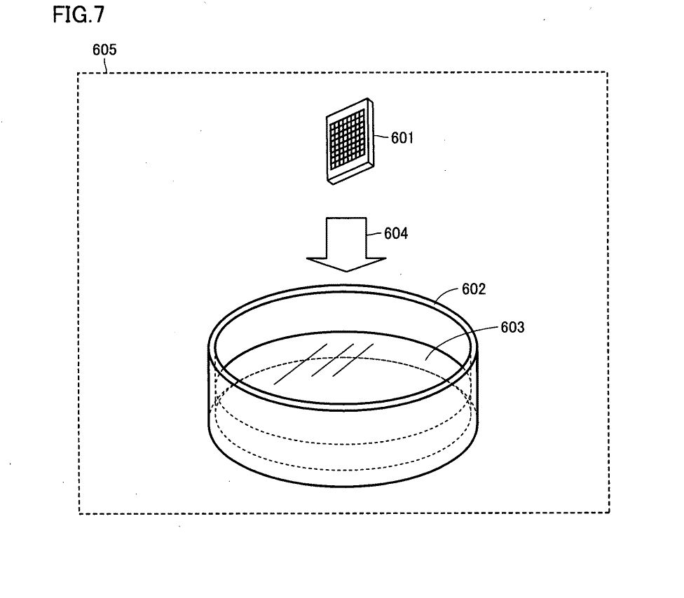

Various bacteria were used as cells, and they were each suspended in a PBS buffer (pH=7.4), and then applied onto an agar medium formed within a tray 602 shown in FIG. 7 for predetermined treatment. Thereafter, they were each cultured at 37° C. for 72 hours, and CFUs (Colony Forming Units) were measured. FIG. 7 is a schematic view of an apparatus used for the present example. In FIG. 7, a PBS buffer 603 is introduced into tray 602, and air containing particles produced by a discharge apparatus 601 is blown in a direction indicated by an arrow 604. This test is to be conducted within a box 605. It is to be noted that tray 602 is illustrated larger in FIG. 7 to facilitate understanding of the drawing, and the relative size illustrated in the drawing does not indicate an actual relative size.

In order to examine sterilization performance of an electric discharge to an adhesive bacterium or of a particle produced by the electric discharge in the present example, firstly, Staphylococcus, Enterococcus, Sarcina flava, and Microcuccus roseus were used as the bacteria, and each applied onto an agar medium according to the above method. Further, they were cultured for eight hours (at 37° C.) to form colonies of the bacteria.

Next, as shown in FIG. 7, air containing active positive and negative ions produced from the discharge apparatus as an electric discharge or particles produced by the electric discharge was diffused as indicated by arrow 604 to distribute the ions all over the agar medium. Thereby, exposure to active air was performed. It is to be noted that box 605 for the test had a size of 21×14×14 cm.

Ion concentration was set at about 1000 ions/cm³ for each of the positive and negative ions on the agar medium (the concentration of small ions was measured with critical mobility set at 1 cm²/V.cm), and ozone concentration was less than 0.01 ppm. The box for the test does not have a fan inside, and exposure to the ions is performed by natural convection and natural diffusion.

Subsequently, cultivation was performed at 37° C. for 72 hours in the above test to observe CFUs and the states.

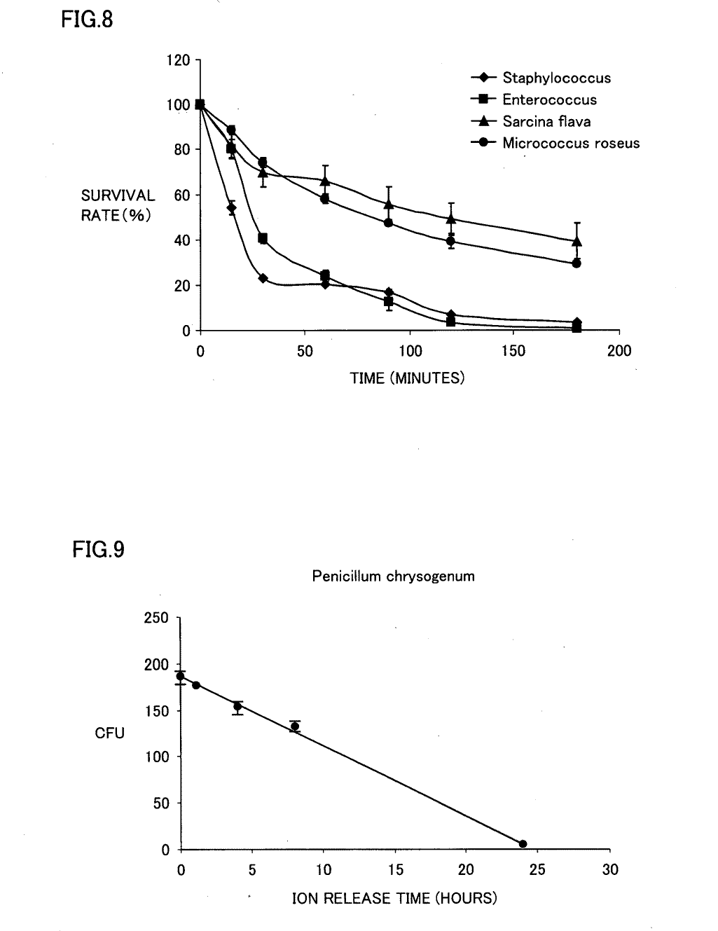

When the positive and negative ions were released to the bacteria in the above test, the CFUs obtained after the cultivation decreased as the release time increased. FIG. 8 shows the result. In FIG. 8, the axis of ordinate represents survival rate of a bacterium, and the axis of abscissa represents time to release the positive and negative ions. It is clear from the result shown in FIG. 8 that the positive and negative ions have an effect of sterilizing the adhesive bacteria.

The above result can be explained by a mechanism as described below. It is likely that the bacterium applied on the agar medium is exposed on the surface of the agar medium as a single substance at first, and when the bacterium comes into contact with the ions in the air, its cell membrane is destroyed and a protein within the cell flows out. This outflow of the protein seems to cause malfunction of the membrane, leading to inactivation (sterilization) of the bacterium. FIG. 8 appears to show the result of the above actions.

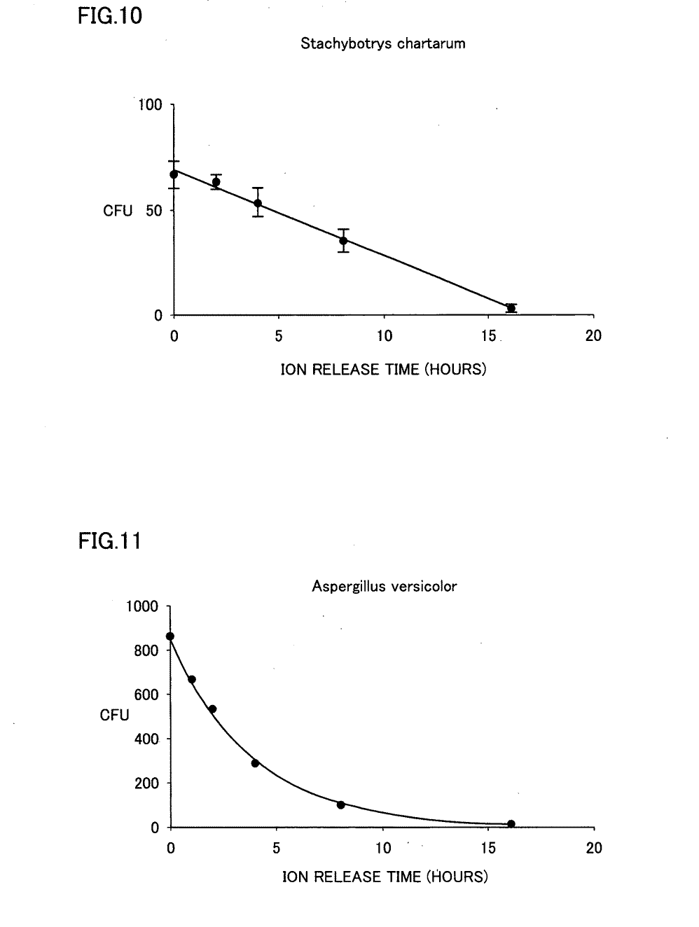

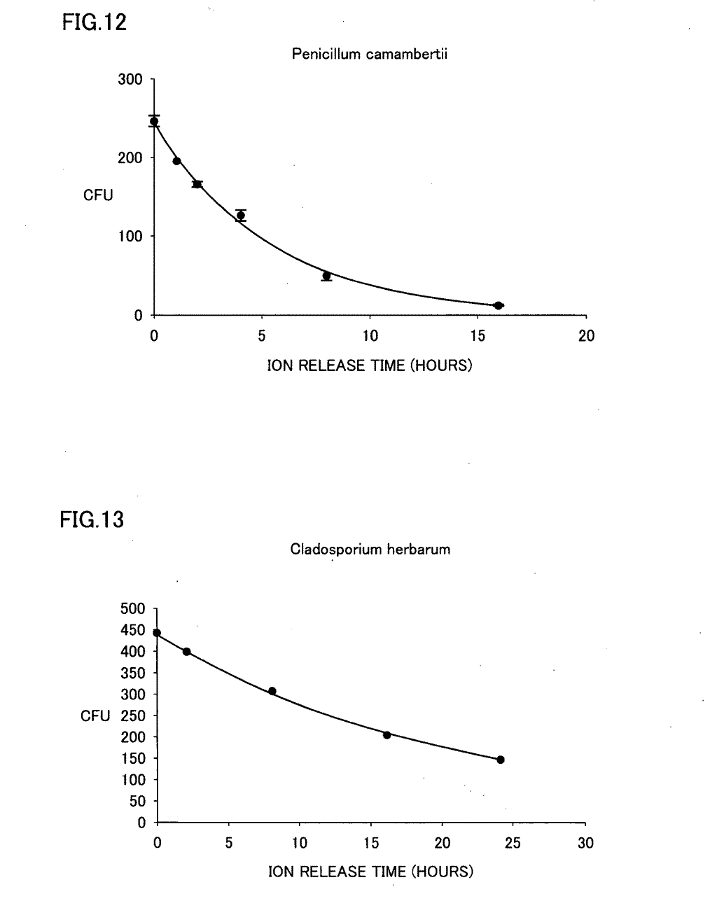

Similarly, in order to examine sterilization performance against a mold, the above experiment was performed using Penicillum chrysogenum, Stachybotrys chartarum, Aspergillus versicolor, Penicillum camambertii, Cladosporium herbarum (a black mold). As a result, it has become clear also for the molds as shown in FIGS. 9 to 13 that, when the ions are released to the molds, the CFUs obtained after the cultivation decreased as the release time increased.

Further, since a mold is a fungus that forms spores resistant to thermal or physical impact, there is a concern that, when a mold starts forming spores, the spores may block the ions and interfere with the degradation of a protein of a fungus according to the present invention. Consequently, as the molds frequently seen in the general living environment, fungi of Aspergillus versicolor and Cladosporium herbarum were each cultured on a petri dish to form spores, and then the ions were released to the spores for four hours. Thereafter, it was examined what change occurred.

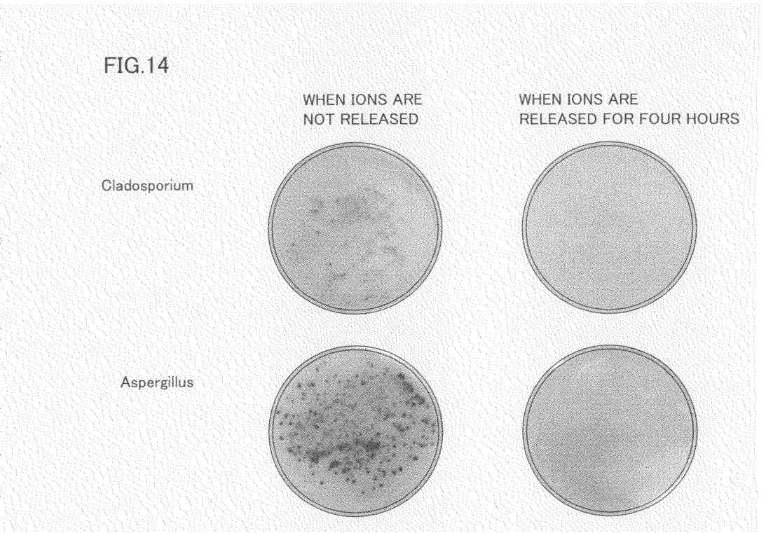

As a result, as shown in FIG. 14, it has been found that ion release can inhibit further formation of the spores and eliminate colonies of the molds. Thus, similar examination was performed also on the molds other than Cladosporium, and it has been found that inhibition of spore formation and elimination of colonies were similarly observed as shown in Table 1. In view of these results, it is understood that the ions have an effect of sterilizing both Gram positive cocci and molds as adhesive bacteria and fungi.

TABLE 1

| Mold | Effect |
|---|---|
| Aspergillus versicolor | +++ |
| Penicillum chrysogenum | +++ |
| Cladosporium herbarum | +++ |
| Stachybotrys chatarum | ++ |
| Penicillum camambertii | ++ |
| Mucor sp. | +++ |
| Alternaria alternata | +++ |

It is to be noted that, in Table 1, the mark +++ (highly effective) indicates that inhibition of spore formation was increased by not less than 80% as compared with the case where no ions were released, the mark ++ (moderately effective) indicates that inhibition of spore formation was increased by less than 80% and not less than 50% as compared with the case where no ions were released, and the mark + (slightly effective) indicates that inhibition of spore formation was increased by less than 50% as compared with the case where no ions were released.

### Example 4

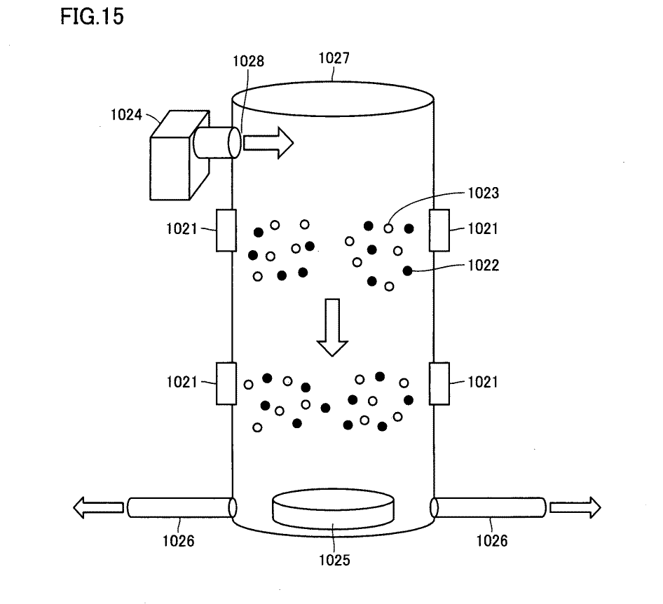

Referring to FIG. 15, the present example will be described. FIG. 15 is a schematic view of an apparatus used in the present example. Four discharge apparatuses 1021 are mounted and fixed within an acrylic cylindrical closed container 1027 having an inner diameter of 140 mm and a length of 500 mm. An inlet 1028 spraying a solution containing an antigenic protein is provided on one side of the container, and a collection receptacle 1025 for the solution containing an antigenic protein is provided on the other side of the container. Further, a gas outlet 1026 for degassing is provided on the bottom of the container.

Specifically, in the apparatus shown in FIG. 15, while the antigenic protein sprayed from inlet 1028 is falling down by gravity to collection receptacle 1025, it is exposed to a positive ion 1022 and a negative ion 1023 supplied from discharge apparatuses 1021 provided within the container, and subjected to the actions of the both ions.

Reduction in reactivity of antigenic proteins Cry j 1 and Cry j 2 extracted from cedar pollens toward their monoclonal antibodies was examined in a case where ion producing devices were not activated after the antigenic protein was sprayed by a nebulizer 1024, and in a case where a voltage of 3.3 kV to 3.7 kV as a peak-to-peak voltage across electrodes was applied to the devices to deliver positive and negative ions, and the positive and negative ions are released to have a concentration within the cylindrical closed container in a range of 11550 ions/cm³ to 12050 ions/cm³, as the total number of the positive and negative ions.

Specifically, 50 μl of each of Cry j 1 and Cry j 2 treated with the ions and Cry j 1 and Cry j 2 not treated with the ions, diluted to 0.1 μg/ml with a sodium hydrogen carbonate buffer (bicarbonate buffer), was applied to each well of respective 96-well plates for ELISA. After the plates were washed three times with a washing buffer, 300 μl of a blocking buffer was applied, and the plates were allowed to stand overnight at 4° C.

After standing overnight, the plates were washed three times, and 50 μl of each of anti-Cry j 1 and Cry j 2 rabbit antibodies diluted 1000 times with (3% skim milk+1% BSA)/PBST was applied to each corresponding well. Thereafter, the plates were washed three times, and 50 μl of HRP-labeled anti-rabbit IgE diluted 1500 times with (3% skim milk+1% BSA)/PBST was applied to each well and the plates were allowed to stand for one hour.

After standing for one hour, the plates were washed three times, and 50 μl of a substrate solution (consisting of 500 μl of ABTS (20 mg/ml), 10 μl of 30% hydrogen peroxide solution, 1 ml of 0.1 M citric acid (pH: 4.2), and 8.49 ml of distilled water) was applied to each well. The plates were allowed to stand until color was developed in a light-shielded condition. Fluorescence intensity was measured by a spectrophotometer (ARVO (trademark) SX). The same reagent as described above was used for each sample, unless otherwise noted.

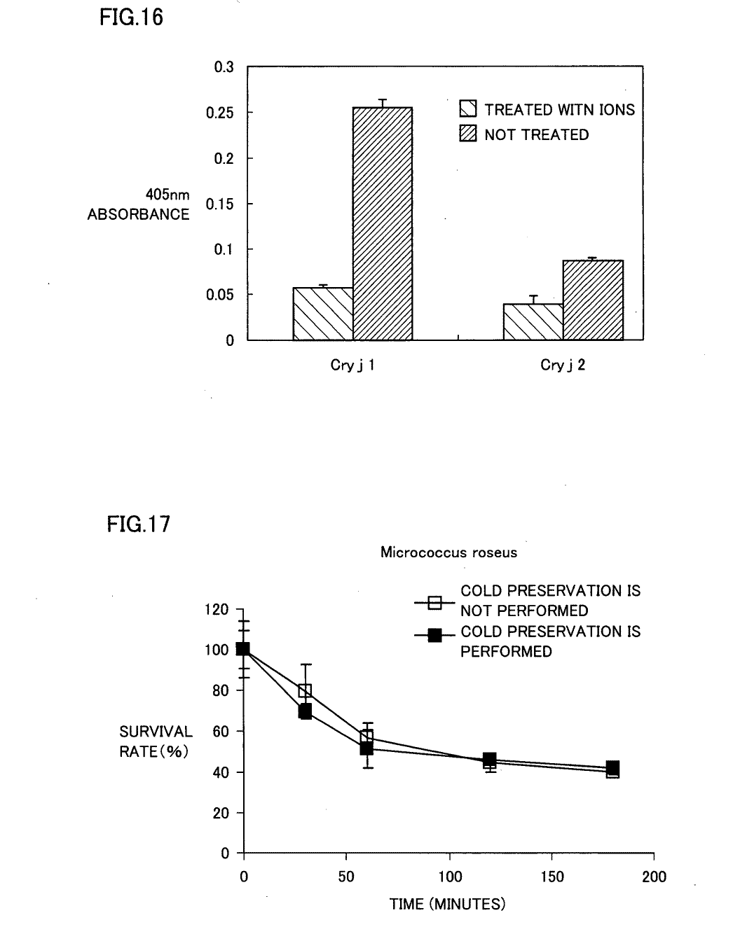

FIG. 16 shows the result obtained above. FIG. 16 shows reactivity of Cry j 1 and Cry j 2 toward their antibodies when treated and not treated with the ions.

As shown in FIG. 16, through the comparison between the case where the discharge apparatuses are not activated (that is, Cry j 1 not treated with the ions and Cry j 2 not treated with the ions) and the case where the positive and negative ions are applied in an atmosphere in which they are released to have a concentration in a range of 11550 ions/cm³ to 12050 ions/cm³, as the total number of the positive and negative ions (that is, Cry j 1 treated with the ions and Cry j 2 treated with the ions), it was confirmed that the reactivity (binding property) of antigenic proteins Cry j 1 and Cry j 2 toward their monoclonal antibodies was significantly reduced when they were treated with the ions. Specifically, it was found that the reactivity of Cry j 1 treated with the ions toward its monoclonal antibody was reduced about one fifth, as compared with the reactivity of Cry j 1 not treated with the ions, and the reactivity of Cry j 2 treated with the ions toward its monoclonal antibody was also reduced about half, as compared with the reactivity of Cry j 2 not treated with the ions.

In this manner, it has become clear that a property or function of a substance, specifically a cell, is changed by the method of the present invention.

### Example 5

Bacteria have self-repairing capacity. It is known that, when sterilization treatment is not satisfactory (for example, due to shortage of time to emit ultraviolet light or lower dose of an agent), bacteria revive and start growing again.

For this reason, irreversibility of bacteria inactivation by releasing positive and negative ions was examined. Bacteria used for the examination were Enterococcus malodoratus, Staphylococcus chromogenes, Micrococcus roseus, and Sarcina flava.

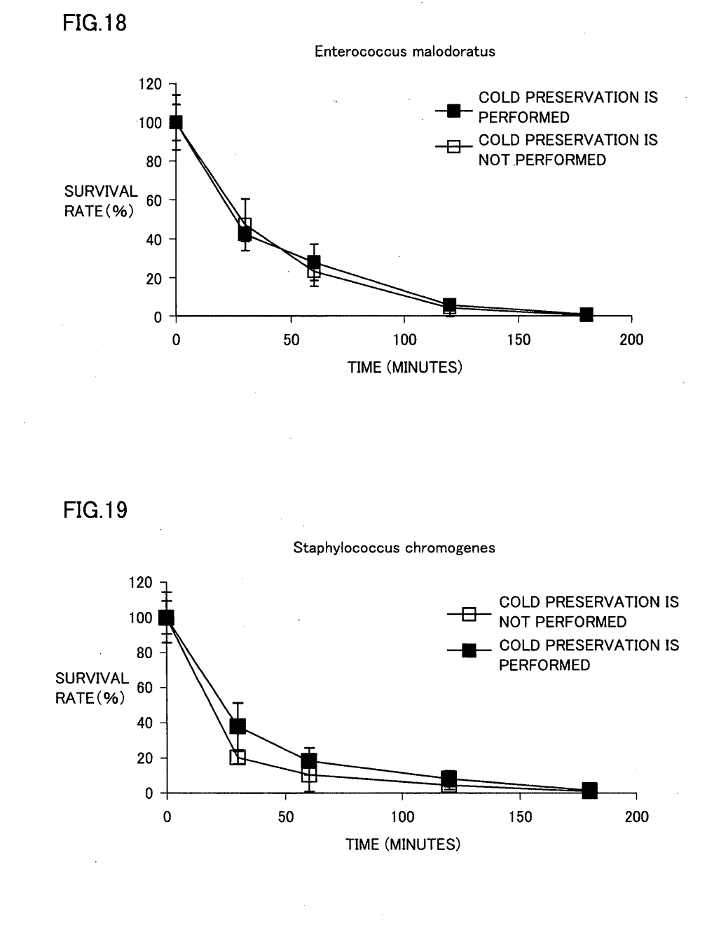

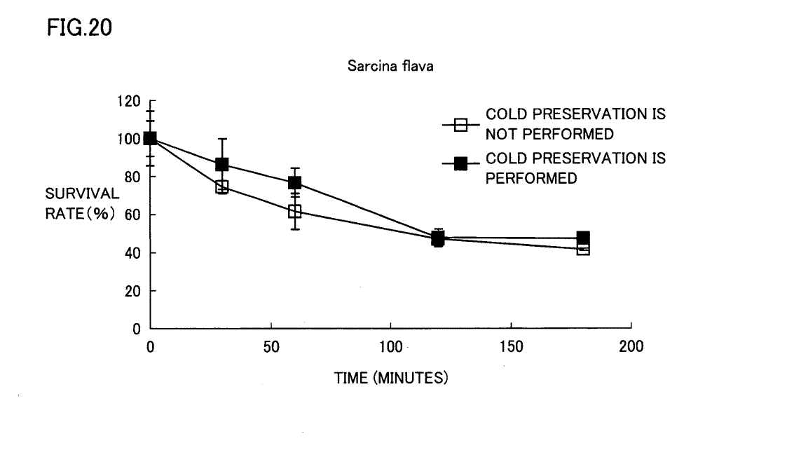

A bacterium was adhered to a medium, and positive and negative ions were released for 90 minutes. The bacterium treated with the ions was cold-preserved at 4° C. for three days. This technique is known as cold delay, which provides recovery time to the bacterium. Change over time in the number of each bacterium surviving after ion release when cold preservation was performed and not performed was measured. FIGS. 17 to 20 show the results. For all of the four bacteria, no significant difference in change over time was confirmed between when cold preservation was performed and when it was not performed, and recovery of the bacteria by cold preservation was not observed.

Further, a bacterium was adhered to a medium, and positive and negative ions were released for 90 minutes. Thereafter, the bacterium was cultured in an incubator at 37° C. for 48 hours to produce colonies of the bacterium. The bacterium was further cultured at 37° C. for 21 days, and then it was examined whether a new colony was produced or not. As a result, production of a new colony was not confirmed even after the cultivation for 21 days. Therefore, recovery of the bacterium was not observed even in a growing environment.

To examine a deterioration effect on a medium by ion release, a bacterium was adhered to the medium, and positive and negative ions were released for 90 minutes. Thereafter, the bacterium was transferred to a medium not treated with the ions, and recovery of the bacterium was examined. However, recovery of the bacterium was not observed.

As described above, it has been found that the method of the present invention can fragment a cell membrane protein on the surface of a bacterium, cause a loss of self-repairing capacity of the bacterium, and eliminate a biofunction of the bacterium, specifically completely kill the bacterium.

1. A method of changing a property or function of a substance by fragmenting a protein contained in the substance floating in a gas.

2. The method according to claim 1, wherein said substance is a granular substance, a microorganism, or a cell.

3. The method according to claim 1, wherein a particle having reactivity is applied to said substance during said fragmenting.

4. The method according to claim 3, wherein said particle includes a charged particle or an excited particle, and when the particle produced by an electric discharge is applied, either one or both of said charged particle and said excited particle are released to said substance.

5. The method according to claim 4, wherein said charged particle includes a positive ion and a negative ion, said positive ion being H⁺(H₂O)ₙ (where n is a natural number), and said negative ion being O₂⁻(H₂O)ₘ (where m is a natural number).

6. The method according to claim 3, wherein the particle produced by said electric discharge includes a hydroxy radical.

7. The method according to claim 1, wherein, during said fragmenting, an electric discharge or a particle produced by the electric discharge is applied to said substance, or both are simultaneously applied to said substance.

8. The method according to claim 7, wherein said particle includes a charged particle or an excited particle, and when the particle produced by the electric discharge is applied, either one or both of said charged particle and said excited particle are released to said substance.

9. The method according to claim 8, wherein said charged particle includes a positive ion and a negative ion, said positive ion being H⁺(H₂O)ₙ (where n is a natural number), and said negative ion being O₂⁻(H₂O)ₘ (where m is a natural number).

10. The method according to claim 9, wherein said positive ion and said negative ion have a total concentration in a range of 10000 ions/cm³ to 1000000 ions/cm³.

11. The method according to claim 7, wherein the particle produced by said electric discharge includes a hydroxy radical.

12. A method of eliminating a biofunction of a cell by applying to said cell either an electric discharge or a particle produced by the electric discharge, or both simultaneously, to fragment a protein contained in said cell.

13. The method of eliminating a biofunction of a cell according to claim 12, wherein the particle produced by the electric discharge includes a charged particle or an excited particle, and when the particle produced by the electric discharge is applied, either one or both of said charged particle and said excited particle are released to said cell.

14. The method of eliminating a biofunction of a cell according to claim 13, wherein said charged particle includes a positive ion and a negative ion, said positive ion being H⁺(H₂O)ₙ (where n is a natural number), and said negative ion being O₂⁻(H₂O)ₘ (where m is a natural number).

15. The method of eliminating a biofunction of a cell according to claim 14, wherein said positive ion and said negative ion have a total concentration in a range of 10000 ions/cm³ to 1000000 ions/cm³.

16. The method of eliminating a biofunction of a cell according to claim 12, wherein the particle produced by said electric discharge includes a hydroxy radical.

17. The method of eliminating a biofunction of a cell according to claim 12, wherein said cell is a cell of a microorganism.

18. The method of eliminating a biofunction of a cell according to claim 12, wherein said applying is performed in a gas.
